(19) United States
(12) Patent Application Publication
(10) Pub. No.: US 2003/0129173 A1
(43) Pub. Date: Jul. 10, 2003

(54) Viability and function of pancreatic islets

(76) Inventors: Ismail H. Al-Abdullah, Miami, FL (US); Camillo Ricordi, Miami, FL (US)

Correspondence Address: VENABLE, BAETJER, HOWARD AND CIVILETTI, LLP, P.O. BOX 34385, WASHINGTON, DC, 20043-9998, US

(21) Appl. No.: 10/292,819

(22) Filed: Nov. 13, 2002

Related U.S. Application Data
(60) Provisional application No. 60/331,532, filed on Nov. 19, 2001.

(51) Int. Cl. A61K 48/00; C12N 5/08

(52) U.S. Cl. 424/93.21; 435/366; 435/406

(57) ABSTRACT

An improved culture medium suitable for culture of pancreatic islets, and methods of use.

This application claims priority to U.S. provisional application No. 60/331,532, filed Nov. 19, 2001, which is hereby incorporated by reference.

### BACKGROUND OF THE INVENTION

1. Field of the Invention

The invention relates to an improved culture medium suitable for culture of pancreatic islets, and methods of use.

2. Background Information

Type I diabetes is a widespread metabolic disorder caused by failure of beta cells of the pancreas to secrete sufficient insulin. Insulin is required for the uptake of glucose in most cell types, and inadequate insulin production causes reduced glucose uptake and elevated blood glucose levels. Without proper treatment, diabetes can be fatal.

The usual treatment for Type I diabetes is periodic administration of exogenous insulin by injection. This has a number of disadvantages, including the discomfort and inconvenience of having to administer medication regularly by injection, and the necessity for regular careful monitoring of blood glucose levels to determine that the proper dosage of insulin is administered. Even with this approach, strict control of diet is also required to maintain blood glucose levels within a safe range.

In one approach to treating Type I diabetes, pancreatic transplants have been carried out on a limited number of individuals. However, these have had limited success due to the usual drawbacks of transplants, namely the problems of tissue matching and immunological rejection by the recipient and the limited availability of donor organs. For these and other reasons, the possibility of transplanting islet cells into diabetic patients has been of widespread interest. Such cells could be encapsulated in a semipermeable material or contained in a biocompatible device and implanted in patients to secrete insulin as needed for the regulation of glucose levels (see, for example, published PCT application PCT/US92/03327). Because of their low numbers with respect to other pancreatic cell types and the difficulty of isolating and maintaining them in culture until transplantation, this approach has not been as successful as hoped.

Recent progress in achieving successful islet transplantation in clinical practice is partly dependent on the utilization of islets from multiple donors to reverse diabetes in a single diabetic patient. With the current acute shortage of cadaveric organs, it is hard to justify the use of islets isolated from multiple pancreata to treat a single recipient. In addition, even if all the pancreata available from cadaveric donors are used, it would be possible to transplant less than 1% of all patents with diabetes who may benefit from transplantation of insulin producing tissue.

The “Edmonton protocol” (Shapiro et al., N. Engl. J. Med. 1991; 325:1371) is a commonly used method for the isolation of pancreatic islet cells. However, a significant mass of islet yield is lost during the pancreas digestion and islet isolation procedure. In addition, significant islet loss is generally reported after islet culture in serumfree media. Current in vitro culture methods affect islet function and generally result in decreased efficacy following transplantation. The elimination of islet culture in the Edmonton protocol was aimed at avoiding the used of xenogeneic proteins and the loss associated with in vitro culture. For this reason, islet transplantation was classified as an emergency procedure.

There is therefore a critical need to improve the current status of islet culture to maximize recovery of the islets and possibly even allowing repair mechanisms to occur in partially damaged islets following islet isolation and purification procedures. It would be highly desirable to improve the yield, integrity and function of islets from a single pancreas. This would not only allow for improvement of islet recovery, but also make it possible to perform product release tests before transplantation, minimizing or avoiding the loss of islets that has been observed with standard culture procedures.

### SUMMARY OF THE INVENTION

The invention provides an improved culture medium that is particularly useful for isolated pancreatic islet cells, and a method of islet recovery and culture that results in improved islet function and viability. The composition and methods of the invention will result in enhanced transplant capabilities for pancreatic islets, and increased transplantation success. Islet cells can be isolated according to the method of Ricordi et al. (An automated method for the isolation of human pancreatic islets, Diabetes, 1988; 37:413) or other means familiar to those of skill in the art, using the culture medium of the invention, cultured for suitable time periods to maintain or enhance functionality and viability, and introduced into the recipient by any suitable means known in the art, for example, infusion into the liver via a portal vein. Preferably, pancreatic islet cells are cultured in the medium of the invention for periods of 3 hours to 4 weeks, more preferably for 3 hours to 12 days, most preferably for 24 to 48 hours. However, the cells should be able to be cultured using the present compositions and methods for 90 days or even longer.

The compositions and methods of the invention are particularly useful in isolating and culturing islets prior to transplantation to treat diabetes mellitus.

In one embodiment, the invention provides a novel culture medium that differs from the conventionally used medium in that it contains nicotinamide and vitamin E (also known as γ-tocopherol, α-tocopherol or Trolox) and fetal calf serum is replaced with human serum albumin. In this embodiment, the culture medium comprises a base medium suitable for the cultivation of mammalian cells, such as CMRL 1066 (GibcoBRL/Life Technologies, Inc., Gaithersburg, Md., USA), to which effective amounts of nicotinamide, vitamin E and HSA are added. Other equivalent base media may be used in place of CMRL including, but not limited to, Basal Media Eagle (BME), DMEM; DMEM/F12, Medium 199; F-12 (Ham) Nutrient Mixture; F-10 (Ham) Nutrient Mixture; Minimal Essential Media (MEM), Williams' Media E; RPMI 1640, CO₂ independent medium and mixtures thereof. (These formulations are available from Gibco-BRL/Life Technologies, Inc., Gaithersburg, Md. and other commercial sources). Persons of skill in the art will appreciate that many other suitable base media are commercially available, or can be routinely formulated in the laboratory. In general, such base media contain inorganic salts (e.g. NaCl, KCl, NaH₂PO₄, CaCl₂, MgSO₄, Na acetate), naturally occurring amino acids, vitamins, buffers and additional components as may be advantageous (cholesterol, coenzyme A, glucose glutathione, Thymidine, Uridine-5 triphosphate, antibiotics), as needed to support the viability of cells.

In a particularly preferred embodiment the medium additionally comprises (ITS+Premix), insulin, transferrin, selenium, water soluble linoleic acid, sodium pyruvate, zinc sulfate or zinc chloride, Hepes, and GlutaMax-1. This medium is designated Miami medium-1 (MM-1). It has been found that in vitro human islet function, integrity, and viability of islets cultured in MM-1 are substantially improved over that seen when cultured in conventional media.

In another embodiment, the invention provides a method for isolating pancreatic islet cells, wherein the isolation and wash medium includes HSA and vitamin E, and optionally nicotinamide. In a preferred embodiment, the isolation medium consists of RPMI to which 25 mM HEPES (pH 7.4), 0.7% HSA and 10 μM vitamin E have been added. In another embodiment, the invention provides a method for culturing pancreatic islet cells, in particular human pancreatic islet cells, that improves viability and function. The method comprises culturing isolated pancreatic islet cells in the culture medium of the invention. Thus, the invention also provides a method of improving the viability of isolated pancreatic islet cells by culturing the cells in the culture medium of the invention.

In yet another embodiment, the invention provides a method of transplanting pancreatic islet cells, said method comprising isolating pancreatic islet cells from a pancreas, culturing said cells in the culture medium of claim 1, 2 or 3, and introducing said cells into a host. In a particularly preferred embodiment, the host is a patient in need of a transplant of insulin producing cells, for example, a patient having Type I diabetes. Thus, the invention also provides a method for treating diabetes mellitus, particularly Type I diabetes, comprising the steps of isolating pancreatic islet cells, culturing the islet cells in the culture medium of the invention, and transplanting the cells into a patient.

### BRIEF DESCRIPTION OF THE DRAWINGS

FIGS. 1A-1D: Human pancreatic islets cultured for 10 days in MM-1 and CMRL+10% FCS. FIGS. 1A and 1B show Dithizone (DTZ) staining of islets cultured in MM-1 and CMRL cells, respectively. FIGS. 1C and 1D show islet aggregation in MM-1 and CMRL cells, respectively. Photomicrographs (50×) were taken under stereomicroscope.

FIG. 2: Stimulation indices of islets cultured in MM-1 and conventional CMRL-10% FCS.

FIG. 3A: Islet equivalents before and after culture in MM-1.

FIG. 3B: Total tissue volume before and after culture in MM-1.

FIG. 4A: Reduction of total acinar tissue volume from 21 pancreata after culture in MM-1.

FIG. 4B: Comparison of relative volume of IEQ and acinar tissue pre and post culture in MM-1.

### DETAILED DESCRIPTION OF THE INVENTION

A significant mass of islet yield is lost during typical pancreas digestion and islet isolation procedures. In addition, significant loss of islet count after culturing in serum-free media has been reported. The Edmonton protocol initiated islet transplantation as an emergency procedure to avoid the need for xenogeneic proteins and the islet loss associated with the in vitro culture. Improved culture methods are important to maximize islet recovery, allow repair mechanisms, perform product-release testing, and facilitate pooling islets from different marginal donor pancreata. In addition, achieving therapeutic immunosuppressive levels in the recipients before transplantation, obviates the need for the emergency transplantation procedure.

Extensive studies have been carried out to determine the best suitable tissue culture medium for culturing islets. Islets from rat, porcine and human pancreata have been compared and contrasted using CMRL and other culture media. The human islets were well preserved, because their integrity, viability and in vitro ability to respond to glucose were optimum when cultured in CMRL containing 10% FCS.

CMRL was originally described as a serum-free medium that has been found to be superior to other commercially available media, especially when supplemented with ITS+Premix (Fraga, D W, Sabek, O, Hathaway, D K, Gaber, O; Transplantation 1998, 65, 1060) It is noteworthy that culturing islets has advantages and disadvantages. These include the risk of contamination, hypoxia, and the fact that partially purified islets, when cultured, release noxious substances such as nitric oxide and free radicals. Among the advantages is that the presence of serum in the culture medium positively affects insulin secretion and potential proliferation of the cells. Fetal calf serum is routinely used in culture media, but this xenogeneic protein carries the risk of transmitting xenogeneic agents, and the variability between batches has been well documented.

Substantial experimental evidence supports the potential role of nicotinamide (vitamin B3) and vitamin E in the prevention of beta cell damage, because of their anti-apoptotic, cytoprotective, and antioxidant properties. Based on this evidence, we developed a culture medium designated Miami Medium-1 (MM-1) and used it to culture human islets to assess their function, integrity and viability. This medium is a modification of CMRL-1066 with the addition of nicotinamide, vitamin E and human serum albumin. The latter is replaced with FCS. Also added were insulin, transferrin, selenium, water-soluble linoleic acid, sodium pyruvate, zinc sulphate, HEPES and GlutaMax-1.

Abbreviations:

CMRL: standard growth medium available commercially (e.g. GIBCO, Bio Life, Bio Whitaker)

DMEM: Dulbecco/Vogt modified Eagle's (Harry Eagle) minimal essential medium (commercially available)

RPMI: Roswell Park Memorial Institute medium Glutamax-1: Stable L-glutamine from Gibco

Trysolol: Aprotinin (Bayer). Stock solution is 10,000 K.I. units/ml

Materials and Methods

Human pancreata were obtained from heart-beating cadaveric donors, and were preserved on ice for less than 10 hr in University of Wisconsin Solution (“UW”; ViaSpan(BelzerUW) from Bristol-Mayers Squibb Company, Princeton, N.J., USA) before processing for islet isolation Islets were isolated using the automatic method for pancreas digestion (Ricordi C., Lacy, P et al. An automated method for the isolation of human pancreatic islets, Diabetes, 1988; 37:413). Briefly, the pancreatic duct was cannulated and perfused with cold Liberase-HI(Roche Diagnostic corp, Indianapolis, Ind.) dissolved in Hanks Balanced Salt Solution (HBSS) supplemented with 1.6 ml CaCl₂ (100 mg/ml), 8.5 ml of 1 M HEPES, one ampule (2.5 ml) of Human recombinant DNase Pulmozyme® (1 mg/ml, 2500 units) (Genentech, Inc, San Francisco, Calif.). Perfusion was carried out for 5 min at 80 mmHg and subsequently for another 5 min at 180 mmHg. The distended gland was cut into 7-9 pieces, loaded into Ricordi's chamber and subjected to the automated method to digest the tissue. The islets were then separated using the density gradient purification method.

Miami medium-1 (MM-1) is composed of a base solution of CMRL-1066 to which 12.5 mg/L each of insulin and transferrin, 12.5 μg/L selenious acid; 25 mM HEPES (pH=7.0-7.9); 16.7 μM zinc sulphate, 10 mM nicotinamide, 10 μM water soluble vitamin E, 2 mM Glutamax-1; 5 mM Na pyruvate; 0.002% Ciproflaxin IV; 10.7 mg/mL linoleic acid and 0.25% HSA are added. It is noted that nicotinamide can be varied from 1-40 mM (preferably 10 mM) and vitamin E can be varied from 1 μM-20 mM (preferably 10 μM) with good results. On the day of islet culture, sufficient HSA is added to obtain a final concentration of 0.5%. Islets are cultured in this medium at 5% CO₂ and 37° C. for 24 hr and at 22° C. for the remaining time before transplant (generally 48-70 hr).

Islet preparations isolated from 21 pancreata were cultured for 37 hr (range 7.3-65.5 hr) in MM-1 before transplantation, and the islet equivalent (IEQ) pre and post culture was calculated. Initially, islets were cultured at 37° C., 5% CO₂ and then subsequently cultured at 22° C., 5% CO₂ until transplantation.

Assessment of Islet function and Integrity

In vitro islet function was evaluated by glucose challenges using low (2.2 mM) and high (22 mM) glucose. Triplicates of 100 IEQ per well were incubated for 1 hr at 37° C. with 1 ml Krebs-Ringer bicarbonate buffer containing either 2.2 mM or 22 mM glucose in 12-well plate. Insulin responses to glucose challenges were measured using a commercially available ELISA Kit (Mercodia, Minneapolis, Minn.). Stimulation indices were calculated by dividing mean insulin response of high glucose by mean of insulin response to low glucose.

Stimulation indices were calculated by dividing insulin response to high glucose by the response to low glucose. The rate of islet recovery pre and post culture was also calculated by counting IEQ. Dithizone (DTZ) staining was used to assess the integrity and structural morphology of the islets (Latif Z. A., Noel, J., Alejandro R., A simple method of staining fresh and cultured islets. Transplantation 1988, 45; 827). Briefly, 50 mg DTZ was dissolved in 5 ml DMSO then diluted up to 50 ml with HBSS. The solution was filtered with a 0.2 μ filter and used directly to stain the islets. The islets are stained brown.

Islet count pre- and post-culture in MM-1 showed no significant difference when cultured for up to 65.5 hr, indicating that islet integrity is well preserved when cultured in this medium. The recovery rate of islet equivalent isolated from 21 pancreata showed that islet equivalent pre-culture was 41,9865 vs. 36,6287 IEQ post-culture. The slight reduction of islet mass post culture may be due to partial shrinking of the islets.

### EXAMPLE 1

Comparison of Effects of MM-1 and CMRL Media on Pancreatic Islet Cells

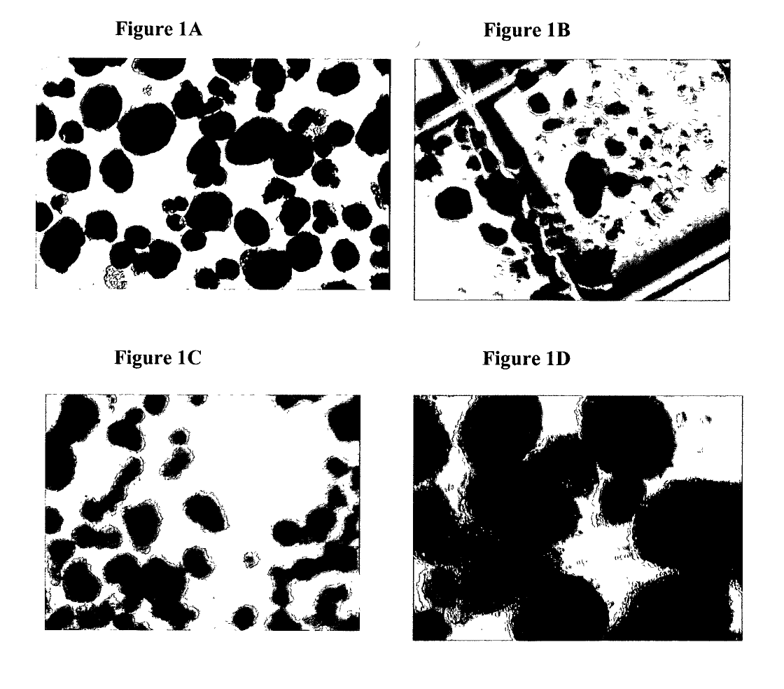

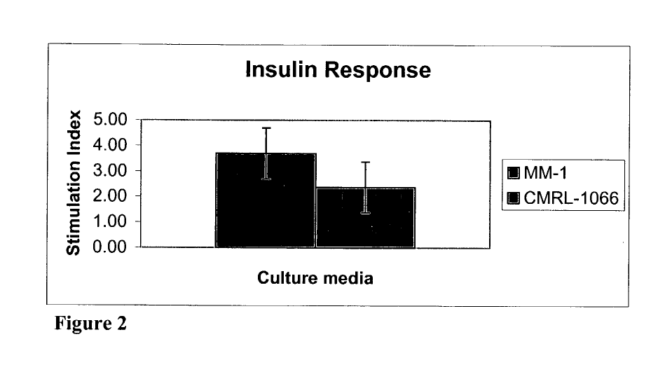

Islets isolated from 12 pancreata were cultured in MM-1 (n=5) which is composed of commercially available CMRL-1066 supplemented with ITS+Premix(6.25 mg/l Insulin, 6.25 mg/l transferrin, 6.25 mg/l selenium, 5.35 mg/l water soluble Linoleic acid), 5 mM sodium pyruvate, 2 mM GlutaMax-1, 25 mM HEPES, 16.7 μM zinc sulphate, 10 mM nicotinamide, 10 μM water soluble vitamin E and 0.65% HSA. Islets from the control group were cultured in CMRL-1066 supplemented with the same ingredients except vitamin E, nicotinamide and human serum albumin. The latter was replaced with 10% FCS (n=7) FIGS. 1A-D show a comparison of islet cells cultured in MM-1 and CMRL+10% FCS for 10 days. Note that the DTZ staining was denser in islets cultured in MM-1 (FIG. 1A) compared to the islets cultured in conventional CMRL+10% FCS (FIG. 1B), indicating that insulin secretion is superior. Islet aggregation was seen less in cells cultured in MM-1 (FIG. 1C) than in CMRL+10% FCS (FIG. 1D). Thus, the integrity and morphological characteristics of the islets were better preserved and protected in MM-1 as compared to the islets cultured in conventional medium. Significantly lower aggregation and clusters of islet flock were seen in MM-1 compared to islets cultured in CMRL. The stimulation indices (FIG. 2) were slightly higher for islets cultured in MM-1 (3.7±1.15) than for islets cultured in conventional CMRL+10% FCS (2.37±0.66), p=0.06.

### EXAMPLE 2

Characteristics of Pancreatic Islet Cells Cultured in MM-1

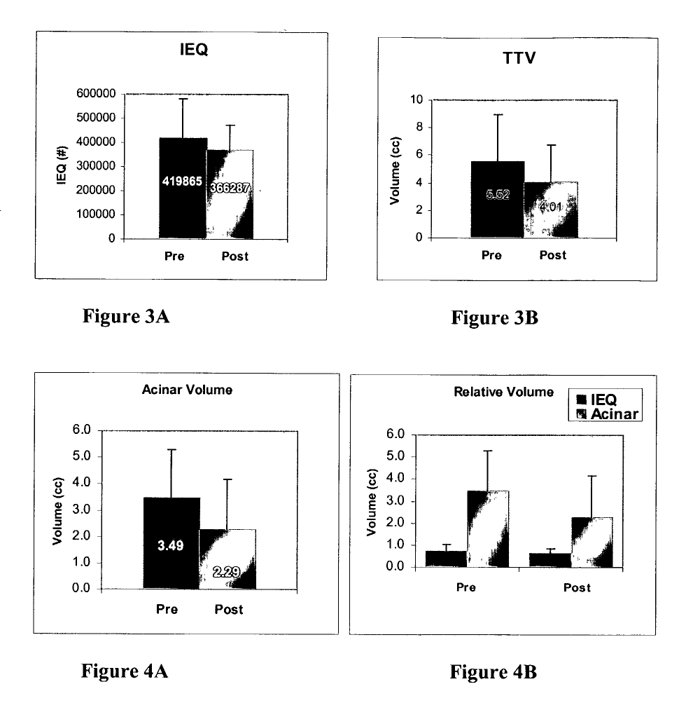

Human islets were isolated from 21 pancreata, and cultured in MM-1 for an average of 37 hr (range 7.3-65.5). The recovery rate of islet equivalent were calculated pre- and post-culture. There was no significant reduction in islet mass (p=0.2). The slight reduction of islet mass post culture (shown in FIG. 3A) is probably due to the partial shrinking of the islets. Note that the total tissue volume (TTV) was reduced post-culture from 5.52±3.4 ml to 4.0±2.8 (p<0.05)(FIG. 3B).

The tissue volume was measured pre- and post-culture in MM-1, and this was subtracted from total IEQ. Islet equivalent (IEQ) was measured using a previously described method (Ricordi, C., Quantitative and qualitative standards for islet isolation assessment in human and large mammals, Pancreas 1991, 6, 242-244.) Tissue volume (TTV) was measured using calibrated 50 ml conical tubing, and the total tissue volume was calculated by subtracting from the total IEQ. FIG. 4A shows acinar volume before and after culture in MM-1. FIG. 4B shows the relative tissue volume of IEQ and acinar tissue present in the islet preparation pre- and post-culture in MM-1. It is evident that acinar volume is reduced, while IEQ remains constant following culture.

### EXAMPLE 3

Culture and Transplantation of Pancreatic Islet Cells into Diabetic Patients

Islets were isolated from twelve donor pancreata using the automatic method of Ricordi et al., and purified by density gradient. Islets were cultured in MM-1 for an average of 34 hours (range 12-65 hours) and subsequently transplanted into the livers of 6 diabetic patients via portal vein (2 donors/patient). Edmonton immunosuppressive protocol (Shapiro et al.) was used to prevent rejection. All nine patients were well and insulin free with excellent islet function up to 270 days following transplantation.

In conclusion, when human islets are cultured in Miami medium-1 (MM-1) for a short period (31 hr-10 days) islet integrity and viability is well preserved, and, most importantly, they function well when transplanted into diabetic recipients, reversing the diabetes.

In describing preferred embodiments of the present invention, specific terminology is employed for the sake of clarity. However, the invention is not intended to be limited to the specific terminology so selected. It is to be understood that each specific element includes all technical equivalents, which operate in a similar manner to accomplish a similar purpose.

The embodiments illustrated and discussed in the present specification are intended only to teach those skilled in the art the best way known to the inventors to make and use the invention, and should not be considered as limiting the scope of the present invention. The exemplified embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

All patents, published patent applications and other published references cited herein are hereby incorporated by reference.

What is claimed is:

1. A cell culture medium comprising a base medium suitable for the culture of mammalian cells, said medium comprising 1 μM-20 mM water soluble vitamin E and 140 mM nicotinamide.

2. The cell culture medium of claim 1 wherein the base medium is CMRL 1066, without L-glutamine and phenol red; or RPMI.

3. The cell culture medium of claim 2, further comprising:
2 mM Glutamax-1;
2.5 mM HEPES;
mM sodium pyruvate;
0.00048% zinc sulfate or equivalent zinc chloride;
12.5 mg/L Insulin;
12.5 mg/L transferrin;
12.5 μg/L selenious acid;
10.7 mg linoleic acid; and
0-10% CO₂,
and having a pH between 7.0 and 7.9.

4. The culture medium of claim 1, 2 or 3 that additionally includes human serum albumin at a final concentration between 0.5% and 10%.

5. A method of culturing isolated pancreatic islet cells, said method comprising culturing said islet cells in the culture medium of one of claims 1-4.

6. The method of claim 5 wherein the cells are cultivated for a time period of 3 hours to 12 days.

7. The method of claim 6 wherein the cells are cultivated in 5% CO₂ for 24 hours at 37° C., and thereafter are cultivated at 22° C.

8. A method of transplanting pancreatic islet cells, said method comprising
a) isolating pancreatic islet cells from a pancreas,
b) culturing said cells in the culture medium of one of claims 1-4, and
c) introducing said cells into a host.

9. The method of claim 8 wherein the cells are obtained from a human pancreas.

10. The method of claim 8 wherein the culture medium of claim 2 is used in step a).

11. The method of claim 8 wherein the cells are cultured for a time period between 24 and 48 hours.

12. The method of claim 8 wherein the cells are maintained in the culture medium for a period of between 0 and 4 weeks.

13. The method of claim 8 wherein the cells are introduced into the liver of the host via a portal vein.

14. The method of claim 8 wherein step b) is carried out at 37° C., 5% CO₂ for 24 hours and subsequently at 22° C. prior to step c).

15. The method of claim 8 wherein the host is diabetic.

16. The method of claim 15 wherein the host is a human.

17. A method of maintaining the viability and function of an isolated animal cell or population of cells, said method comprising culturing said cell(s) in the medium of one of claims 1-4.

18. The method of claim 17, wherein said cell(s) are pancreatic islet cells.

19. The method of claim 17 or 18 wherein said cell(s) are mammalian cells.

20. The method of claim 19 wherein said cell(s) are human cells.

21. A method of treating a patient having diabetes mellitus comprising the steps of
a) isolating pancreatic islet cells from a pancreas,
b) culturing said cells in the culture medium of one of claims 1-4, and
c) introducing said cells into said patient.
